(19) United States
(12) Patent Application Publication
(10) Pub. No.: US 2018/0344252 A1
(43) Pub. Date: Dec. 6, 2018

(54) DETECTING BODY PART ACTIVITY USING THE INTERNAL THORACIC VEIN

(71) Applicant: CARDIAC PACEMAKERS, INC., St. Paul, MN (US)

(72) Inventors: QI AN, BLAINE, MN (US); PRAMODSINGH HIRASINGH THAKUR, WOODBURY, MN (US); VIKTORIA A. AVERINA, SHOREVIEW, MN (US); JAMES O. GILKERSON, STILLWATER, MN (US); G. SHANTANU REDDY, MINNEAPOLIS, MN (US); RAMESH WARIAR, BLAINE, MN (US); JEFFREY E. STAHMANN, RAMSEY, MN (US); KEITH R. MAILE, NEW BRIGHTON, MN (US)

(73) Assignee: CARDIAC PACEMAKERS, INC., St. Paul, MN (US)

(21) Appl. No.: 15/993,113

(22) Filed: May 30, 2018

Related U.S. Application Data
(60) Provisional application No. 62/513,084, filed on May 31, 2017.

(51) Int. Cl. A61B 5/00; A61N 1/36

(52) U.S. Cl. CPC A61B 5/6876; A61B 5/686; A61N 1/37211; A61B 5/0031; A61N 1/36135

(57) ABSTRACT

IMD devices, systems, and treatment methods are discussed and disclosed. An IMD having oscillatory sensors for placement in an internal thoracic vein (ITV) of a patient may be employed to detect activity of a body part, determining a status of the body part or patient more generally, and then monitor the status of the body part. Therapy decisions may rely on the status of the body part, and information related to such status may be communicated to an external device.

### CROSS REFERENCE TO RELATED APPLICATIONS

The present application claims the benefit of and priority to U.S. Provisional Patent Application Ser. No. 62/513,084, filed May 31, 2017 and titled DETECTING BODY PART ACTIVITY USING THE INTERNAL THORACIC VEIN, the disclosure of which is incorporated herein by reference.

### BACKGROUND

Patient activity and motion of a patient overall or a body part or organ of a patient can be observed to monitor patient status, recognize and treat ailments, and determine whether provided therapy has an intended effect, among other benefits. Implantable and wearable devices are being implemented for a broad array of biomedical applications including diagnosis and/or treatment of sicknesses, diseases, and disorders. Moreover, recent advances and reduced cost, support the expectation that sensing devices will play an increasingly important role in biomedical systems for basic research, clinical diagnostics, and in-vivo therapy and diagnostic purposes. As a result, there has been an increased interest in sensing devices. With such interest, new and alternative methods of detecting and monitoring body parts of a patient are desired, including for use in implantable devices.

### OVERVIEW

The present inventors have recognized that the internal thoracic veins (ITVs) may provide an opportunity for implanting sensing devices to provide activity detection and monitoring of body parts of a patient.

A first non-limiting example takes the form of a method of treating a patient using an implantable medical device (IMD), the method comprising detecting activity of a body part of the patient using at least one oscillatory sensor of the IMD, the at least one oscillatory sensor located in an internal thoracic vein (ITV) of the patient, obtaining a status of a physiological parameter using the IMD based on the activity of the body part, monitoring the status of the physiological parameter using the IMD, and presenting an indication of the status.

Additionally or alternatively a second non-limiting example takes the form of a method as in the first non-limiting example wherein the indication is presented using the IMD.

Additionally or alternatively a third non-limiting example takes the form of a method as in the first non-limiting example further comprising communicating the status to an external device, and presenting an indication of the status using the external device.

Additionally or alternatively a fourth non-limiting example takes the form of a method as in the first to third non-limiting examples wherein the body part is a heart.

Additionally or alternatively a fifth non-limiting example takes the form of a method as in the fourth non-limiting example wherein the activity is a motion of the heart and the at least one oscillatory sensor is a microphone.

Additionally or alternatively a sixth non-limiting example takes the form of a method as in the fourth non-limiting example wherein the activity is motion of the heart and the at least one oscillatory sensor is an accelerometer.

Additionally or alternatively a seventh non-limiting example takes the form of a method as in the fifth to sixth non-limiting examples wherein the physiological parameter is heart sound.

Additionally or alternatively an eighth non-limiting example takes the form of a method as in the seventh non-limiting example wherein monitoring the status of the heart sound includes observing a fourth heart sound (S₄) from the heart sound.

Additionally or alternatively a ninth non-limiting example takes the form of a method as in the seventh non-limiting example wherein monitoring the status of the heart sound includes observing a heart murmur from the heart sound.

Additionally or alternatively a tenth non-limiting example takes the form of a method as in the ninth non-limiting example wherein a second oscillatory sensor and a third oscillatory sensor are also implanted inside the patient and the at least one oscillatory sensor, the second oscillatory sensor, and the third oscillatory sensor are used to triangulate a source of the heart murmur.

Additionally or alternatively an eleventh non-limiting example takes the form of a method as in the seventh non-limiting example wherein monitoring the status of the heart sound includes observing at least one of a cannon wave and a cannon sound.

Additionally or alternatively a twelfth non-limiting example takes the form of a method as in the eleventh non-limiting example wherein monitoring the status of the heart sound further includes determining AF of the heart based on observing the at least one of the cannon wave and the cannon sound, and calculating a degree of the AF.

Additionally or alternatively a thirteenth non-limiting example takes the form of a method as in the first to third non-limiting examples wherein the body part is a lung.

Additionally or alternatively a fourteenth non-limiting example takes the form of a method as in the thirteenth non-limiting example wherein the activity is breathing and the at least one oscillatory sensor is a microphone.

Additionally or alternatively a fifteenth non-limiting example takes the form of a method as in the thirteenth non-limiting example wherein the activity is breathing and the at least one oscillatory sensor is an accelerometer.

Additionally or alternatively a sixteenth non-limiting example takes the form of a method as in the fourteenth to fifteenth non-limiting examples wherein the physiological parameter includes a respiratory sound.

Additionally or alternatively a seventeenth non-limiting example takes the form of a method as in the fourteenth to sixteenth non-limiting examples wherein the physiological parameter includes a respiratory interval.

Additionally or alternatively an eighteenth non-limiting example takes the form of a method as in the fourteenth to seventeenth non-limiting examples wherein the physiological parameter includes a respiratory amplitude.

Additionally or alternatively a nineteenth non-limiting example takes the form of a method as in the fourteenth to eighteenth non-limiting examples wherein the physiological parameter includes a frequency of oscillation indicating wheezing.

Additionally or alternatively a twentieth non-limiting example takes the form of a method as in the fourteenth to nineteenth non-limiting examples wherein the physiological parameter includes a frequency of oscillation indicating rales.

Additionally or alternatively a twenty-first non-limiting example takes the form of a method as in the fourteenth to twentieth non-limiting examples wherein the physiological parameter includes a frequency or pattern of vibration indicating snoring.

Additionally or alternatively a twenty-second non-limiting example takes the form of a method as in the fourteenth to twentieth non-limiting examples wherein the physiological parameter includes a frequency or pattern of oscillation indicating rhonchi.

Additionally or alternatively a twenty-third non-limiting example takes the form of a method as in the fourteenth to twenty-second non-limiting examples wherein monitoring the status of the physiological parameter includes observing a pathological asymmetrical respiratory pattern from the physiological parameter.

Additionally or alternatively a twenty-fourth non-limiting example takes the form of a method as in the fourteenth to twenty-third non-limiting examples wherein monitoring the status of the physiological parameter includes observing a respiratory distress from the physiological parameter.

Additionally or alternatively a twenty-fifth non-limiting example takes the form of a method as in the twenty-fourth non-limiting example wherein the respiratory distress includes asthma.

Additionally or alternatively a twenty-sixth non-limiting example takes the form of a method as in the twenty-fourth non-limiting example wherein the respiratory distress includes Chronic Obstructive Pulmonary Disease (COPD).

A twenty-seventh non-limiting example takes the form of a method of treating a patient comprising detecting activity of a body part of a patient using an oscillatory sensor disposed on a lead which is placed in an internal thoracic vein (ITV) of the patient.

Additionally or alternatively a twenty-eighth non-limiting example takes the form of a method as in the twenty-seventh non-limiting example wherein the lead and the oscillatory sensor are in the right ITV.

Additionally or alternatively a twenty-ninth non-limiting example takes the form of a method as in the twenty-seventh non-limiting example wherein the lead and the oscillatory sensor are in the left ITV.

Additionally or alternatively a thirtieth non-limiting example takes the form of a method as in the twenty-seventh to twenty-ninth non-limiting examples wherein the oscillatory sensor is an accelerometer.

Additionally or alternatively a thirty-first non-limiting example takes the form of a method as in the twenty-seventh to twenty-ninth non-limiting examples wherein the oscillatory sensor is a microphone.

Additionally or alternatively a thirty-second non-limiting example takes the form of a method as in the twenty-seventh to twenty-ninth non-limiting examples wherein the oscillatory sensor is a hydrophone.

Additionally or alternatively a thirty-third non-limiting example takes the form of a method as in the twenty-seventh to thirty-second non-limiting examples wherein the body part is a heart and the activity is motion of the heart.

Additionally or alternatively a thirty-fourth non-limiting example takes the form of a method as in the twenty-third non-limiting example wherein a heart sound is observed from the motion of the heart.

Additionally or alternatively a thirty-fifth non-limiting example takes the form of a method as in the thirty-third non-limiting example wherein at least one of a cannon wave and a cannon sound is observed from the motion of the heart.

Additionally or alternatively a thirty-sixth non-limiting example takes the form of a method as in the thirty-fourth to thirty-fifth non-limiting examples further comprising determining atrial fibrillation (AF) of the heart based on at least one of the heart sound, the cannon wave, and the cannon sound.

Additionally or alternatively a thirty-seventh non-limiting example takes the form of a method as in the thirty-third non-limiting example wherein valvular stenosis is observed from the motion of the heart.

Additionally or alternatively a thirty-eighth non-limiting example takes the form of a method as in the thirty-third non-limiting example wherein valve regurgitation is observed from the motion of the heart.

Additionally or alternatively a thirty-ninth non-limiting example takes the form of a method as in the twenty-seventh to thirty-second non-limiting examples wherein the body part is a lung and the activity is breathing.

Additionally or alternatively a fortieth non-limiting example takes the form of a method as in the thirty-ninth non-limiting example wherein a respiratory interval is observed from the breathing.

Additionally or alternatively a forty-first non-limiting example takes the form of a method as in the thirty-ninth non-limiting example wherein a respiratory amplitude is observed from the breathing.

Additionally or alternatively a forty-second non-limiting example takes the form of a method as in the fortieth to forty-first non-limiting examples further comprising determining pneumonia of the lung based on at least one of the respiratory interval and the respiratory amplitude, and monitoring the pneumonia.

Additionally or alternatively a forty-third non-limiting example takes the form of a method as in the fortieth to forty-first non-limiting examples further comprising determining respiratory distress of the lung based on at least one of the respiratory interval and the respiratory amplitude, and monitoring the respiratory distress.

Additionally or alternatively a forty-fourth non-limiting example takes the form of a method as in the thirty-ninth non-limiting example wherein wheezing is observed from the breathing.

Additionally or alternatively a forty-fifth non-limiting example takes the form of a method as in the thirty-ninth non-limiting example wherein rales is observed from the breathing.

Additionally or alternatively a forty-sixth non-limiting example takes the form of a method as in the thirty-ninth non-limiting example wherein snoring is observed from the breathing.

Additionally or alternatively a forty-seventh non-limiting example takes the form of a method as in the twenty-seventh to forty-sixth non-limiting examples wherein a second oscillatory sensor and a third oscillatory sensor are also disposed on the lead and the oscillatory sensor, the second oscillatory sensor, and the third oscillatory sensor are used to triangulate a source of the activity.

A forty-eighth non-limiting example takes the form of an implantable medical device comprising a lead having an oscillatory sensor disposed thereon and an implantable canister for coupling to the lead, the implantable canister housing operational circuitry configured to detect activity of a body part of a patient using the oscillatory sensor according to a method as in the twenty-seventh to forty-seventh non-limiting examples.

A forty-ninth non-limiting example takes the form of an implantable medical device system configured for use in a method as in the twenty-seventh to forty-seventh non-limiting examples.

This overview is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1B illustrate a thoracic anatomy including the internal thoracic veins (ITVs) and other parts of the venous structure;

FIG. 2 illustrates a torso in a section view;

FIG. 3A-3B illustrates the ITV and linked vasculature in isolation;

FIG. 4 illustrates an implantable medical device (IMD);

FIG. 5 illustrates a system;

FIG. 6A-6C illustrates thoracic anatomy with the system; and

FIG. 7 is a block flow diagram for an illustrative method.

### DETAILED DESCRIPTION

The internal thoracic vein (ITV), which may also be referred to as the internal mammary vein, is a vessel that drains the chest wall and breasts. There are both left and right internal thoracic veins on either side of the sternum, beneath the ribs. The ITV arises from the superior epigastric vein, accompanies the internal thoracic artery along its course and terminates in the brachiocephalic vein. The present inventors have recognized that the ITV may make a suitable location for placement of an implantable lead for signal sensing capability to allow recognition and discrimination of atrial activity. While much of the following disclosure focuses on the use of the ITV, many of these concepts could also be applied to the internal thoracic arteries, which may sometimes be referenced as the internal mammary arteries. Some additional details related to the use of the ITV for placement of implantable device leads may be found in U.S. patent application Ser. No. 15/667,167, titled IMPLANTATION OF AN ACTIVE MEDICAL DEVICE USING THE INTERNAL THORACIC VASCULATURE, the disclosure of which is incorporated herein by reference.

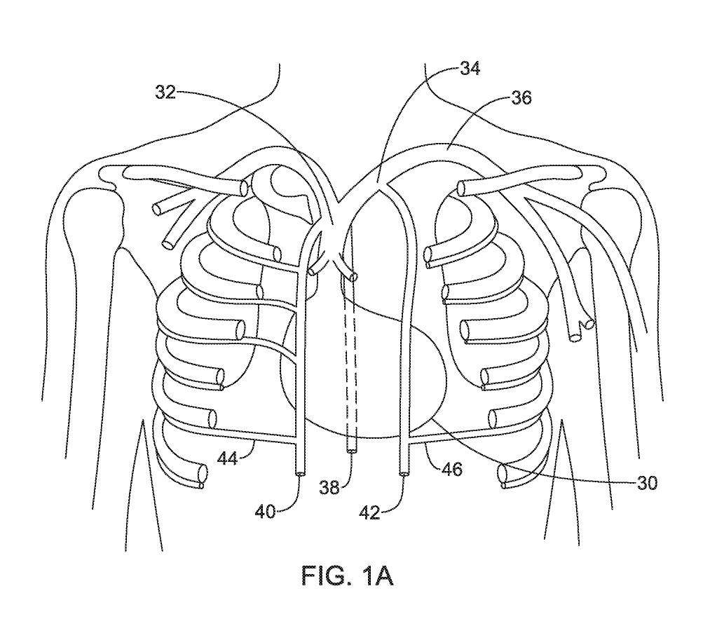

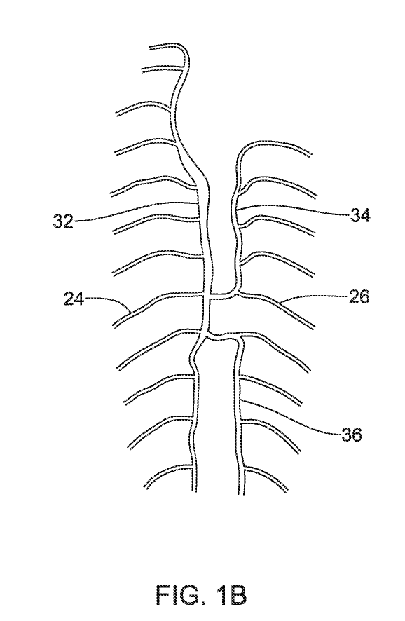

FIG. 1A illustrates the thoracic anatomy including location of the internal thoracic veins (ITVs) 40, 42. A right intercostal vein 44 may couple to the right ITV 40 and a left intercostal vein 46 may couple to the left ITV 42. The right and left intercostal veins 44, 46 may each run along a costal groove on an inferior portion of a rib. An outline of the heart is shown at 30, with the superior vena cava (SVC) shown at 32. The brachiocephalic veins 34 couple to the SVC 32 and extend past various cephalic branches to the subclavian vein 36. The azygos vein is also shown at 38.

As used herein, the “ITV” is the name applied for the vein while it runs beneath the chest, that is, superior to the lower margin of the ribs. Inferior of this location, the blood vessel is referred to (at least in this description) as the superior epigastric vein.

FIG. 1B illustrates the posterior anatomy including placement of the azygos vein, 32, accessory hemiazygos vein 34, and hemiazygos vein 36. The right intercostal vein 24 may run posteriorly along the costal groove on the inferior portion of the right rib and may couple to the azygos vein 32. The left intercostal vein 26 may run posteriorly along the costal groove on the inferior portion of the left rib and may couple to the accessory hemiazygos vein 34. In various embodiments, a left and/or right intercostal vein, at any suitable level of the torso, may be selected and used for implantation of an electrode and lead for use in delivering cardiac therapy. The intercostal veins 24, 26 may be a final implant location for a device or lead, or may provide an avenue for implantation of a device or lead in another part of the anatomy such as in the ITV, the mediastinum, and/or the azygos vein 32, hemiazygos vein 36, or accessory hemiazygos vein 34.

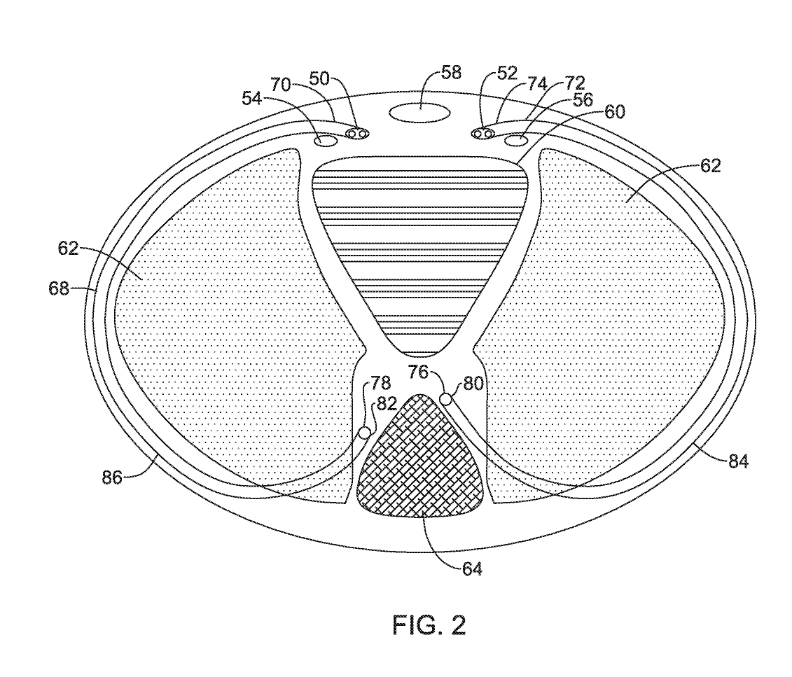

FIG. 2 shows the torso in a section view to highlight the location of various vascular structures. More particularly, in the example, the left and right ITV are shown at 50, 52, running parallel to and more central of the internal thoracic arteries 54, 56, on either side of the sternum 58. The heart is shown at 60, with the lungs at 62 and spinal column at 64. The ITV 50, 52 lie beneath the ribs but outside and separate from the pleurae of lungs 62. The ribs are omitted in the drawing in order to show the intercostal veins. The left anterior intercostal vein 68 runs along the inferior portion of a rib and couples to the left ITV 50 at junction 70, forming an ostium at the point where the left anterior intercostal vein 68 flows into the left ITV 50. Additionally, the right intercostal vein 72 runs along the inferior portion of another rib and couples to the right ITV 52 at junction 74, forming an ostium at the point where the anterior intercostal vein 72 flows into the right ITV 52.

An azygos vein and a hemiazygos vein are shown at 76, 78, respectively, running parallel to and on either side, more or less, of the spinal column 64. The azygos vein 76 and the hemiazygos vein 78 also lie beneath the ribs but outside and separate from the pleurae of lungs 62. The left posterior intercostal vein 86 couples to the hemiazygos vein 78 at a junction 82, forming an ostium at the point where the intercostal vein 86 flows into the hemiazygos vein 78. Additionally, the right posterior intercostal vein 84 couples to the azygos vein 76 at a junction 80, forming an ostium at the point where the intercostal vein 86 flows into the azygos vein 76.

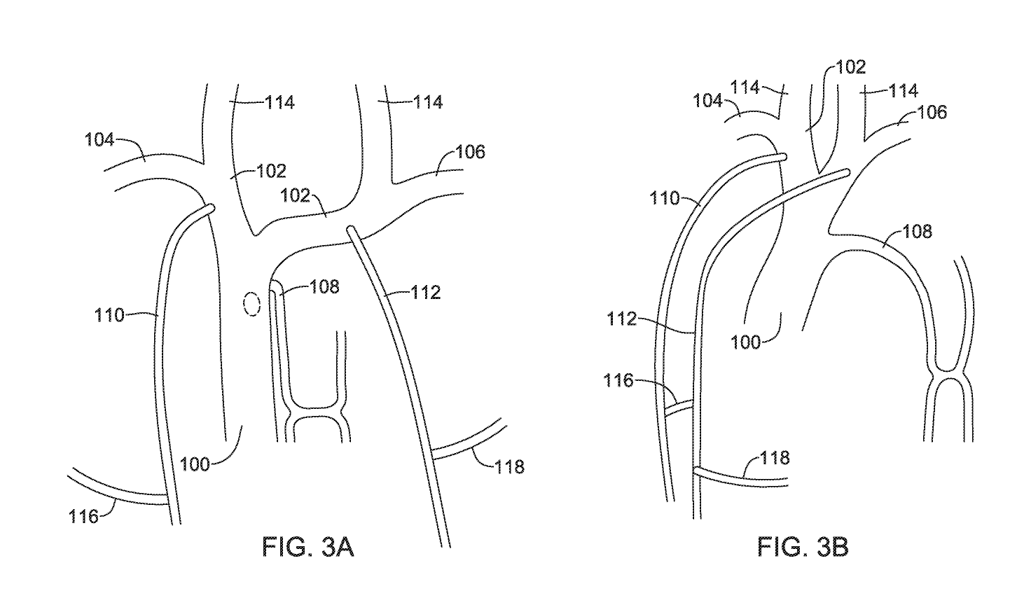

FIGS. 3A-3B show the ITV and linked vasculature in isolation. FIG. 3A is an anterior view of selected portions of the venous structure of the upper torso, and FIG. 3B is a lateral view of the same. The SVC is shown at 100, with the brachiocephalic veins 102 splitting at the upper end of the SVC. The right subclavian vein is at 104, and the left subclavian vein is at 106. The azygos vein is included in the illustration at 108, extending off the posterior of the SVC 100, and running inferiorly posterior of the heart as can be understood from the lateral view of FIG. 3B.

The right and left ITV are shown at 110, 112. These each branch off at a location that is considered part of the brachiocephalic veins 102. Selected right and left intercostal veins are shown at 116, 118. There are left and right intercostal veins along the lower margin of each of the ribs. In several embodiments the intercostal veins of the 4^{th}, 5^{th}, or 6^{th} ribs are proposed for implantation of a lead with access through the intercostal vein to the ITV. In one example, the intercostal vein of the 6^{th} rib is accessed. In other examples, access may be more superior or inferior than these locations, as desired. These may branch off at a location of the right and left ITV's and continue to run along a costal groove of an inferior portion of a the ribs. The internal jugular veins are also shown at 114.

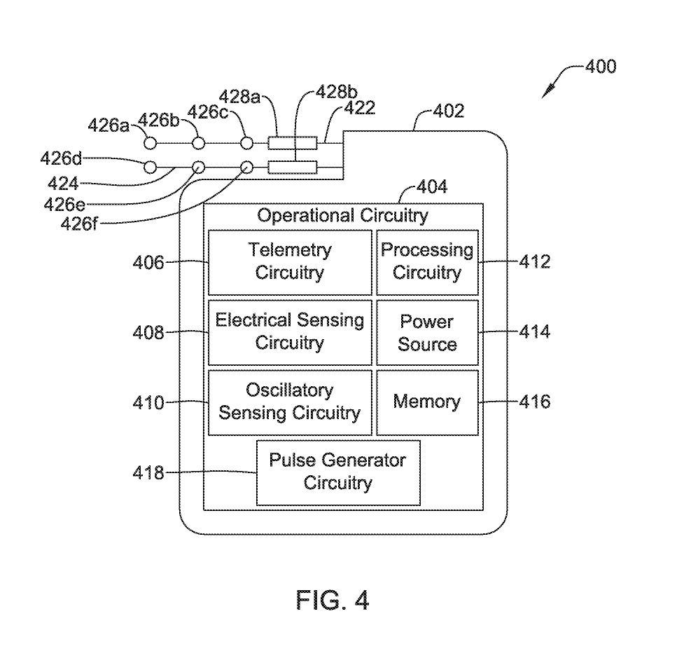

FIG. 4 depicts an illustrative implantable medical device (IMD) 400 that may be implanted into a patient and may detect activity of a body part of the patient. For example, IMD 400 may be an implantable cardioverter defibrillator (ICD). As can be seen in FIG. 4, the IMD 400 may have a housing 402 that encases operational circuitry 404 or electronics. Furthermore, in some examples, the housing 402 may also include a header having a bore for securing one or more leads 422 and 424.

In certain embodiments, the housing 402 may be implanted in, for example, a thoracic region of the patient. In other embodiments, the housing may be implanted in the chest cavity, on the chest, or in an abdominal portion of the patient. The housing 402 may generally include any of a number of known materials that are safe for implantation in a human body and may, when implanted, hermetically seal the various components of the IMD 400 from fluids and tissues of the patient's body.

In the example shown in FIG. 4, the operational circuitry 404 or electronics of the IMD 400 may include telemetry circuitry 406, electrical sensing circuitry 408, oscillatory sensing circuitry 410, processing circuitry 412, a power source 414, memory 416, and pulse generator circuitry 418. The IMD 400 may include more or less circuitry and modules, depending on the application. For example, the pulse generator circuitry 418, the electrical sensing circuitry 408 and/or the oscillatory sensing circuitry 410 may include output switches to select one or more electrodes or vectors for outputting electrical pulses and to select one or more sensors for sensing, filtering, amplification and analog-to-digital conversion circuitry to provide a signal to the processing circuitry 412.

In various embodiments, the leads 422, 424 may include electrical wires that conduct electrical signals between electrodes 426A-426F, oscillatory sensors 428A, 428B and one or more circuits located within the housing 402. In some cases, the leads 422, 424 may be connected to and extend away from the housing 402 of the IMD 400. In some examples, the leads 422, 424 are implanted on, within, or adjacent to a heart and/or lungs of a patient or in or on the chest cavity of the patient. In some examples, the leads 422 and/or 424 may be located in an ITV of a patient. One or more leads 422, 424 and/or electrodes 426A-426F and oscillatory sensors 428A, 428B may be placed subcutaneously, outside the ribs, if desired.

According to various embodiments, the one or more electrodes 426A-426F and oscillatory sensors 428A, 428B may be positioned at various locations on the leads 422, 424, and in some cases at various distances from the housing 402. Some leads may only include a single electrode, some leads may only include a single oscillatory sensor (e.g., 428A and 428B), while other leads may include multiple electrodes (e.g., 426A-426C and 426D-426F) and multiple oscillatory sensors. Generally, the electrodes 426A-426F and the oscillatory sensors 428A, 428B are positioned on the leads 422, 424 such that when the leads 422, 424 are implanted within the patient, one or more of the electrodes 426A-426F and one or more of the oscillatory sensors 428A, 428B are positioned to perform a desired function. For example, the one or more of the electrodes 426A-426F and oscillatory sensors 428A, 428B may be positioned subcutaneously and outside of a body part of a patient (e.g., in an ITV) and sense the activity of the body part (e.g., the heart, lungs, muscles, tissue, etc.). In some cases, one or more of the electrodes 426A-426F may be replaced by a different device such as an ultrasound transducer, a temperature sensor, an optical output and/or receiving device, or another oscillatory sensor such as a microphone or hydrophone, an accelerometer, or other active or passive element. Furthermore, although described with respect to FIG. 4 as separate elements, in some cases, the electrodes 426A-426F and the oscillatory sensors 428A, 428B may be combined into dual functioning elements that perform the operations of both the electrodes 426A-426F and the oscillatory sensors 428A, 428B.

In certain embodiments, the telemetry circuitry 406 may be configured to communicate with devices such as sensors, other medical devices such as a leadless cardiac pacemaker (LCP), an ICD, an implantable pulse generator (IPG), or a wearable device such as a cardiac monitor, and/or the like, that are located externally to the IMD 400. Such devices may be located either external or internal to the patient's body. Irrespective of the location, external devices (i.e. external to the IMD 400 but not necessarily external to the patient's body) can communicate with the IMD 400 via telemetry circuitry 406 to accomplish one or more desired functions.

For example, the IMD 400 may communicate information, such as electrode measurements, oscillatory sensor measurements, sensed electrical signals, data, instructions, messages, etc., to an external medical device (e.g. LCP and/or a programmer or a patient's mobile device such as a phone or watch having Bluetooth or other communications capability) through the telemetry circuitry 406. The external medical device may use the communicated measurements, signals, data, instructions, messages, etc., to perform various functions, such as presenting an indication of the status and/or a change in the status of a physiological parameter of a body part of the patient, storing received data, and/or performing any other suitable function. The IMD 400 may additionally receive information such as signals, data, instructions and/or messages from the external medical devices through the telemetry circuitry 406, and the IMD 400 may use the received signals, data, instructions and/or messages to perform various functions, such as detecting activity of a body part of the patient, obtaining oscillatory sensor measurements, obtaining electrode measurements, storing received data, and/or performing any other suitable function.

The telemetry circuitry 406 may be configured to use one or more methods for communicating with external devices. For example, the telemetry circuitry 406 may communicate via radiofrequency (RF) signals (Bluetooth, ISM, or Medradio, for example), inductive coupling, optical signals, acoustic signals, and/or any other signals suitable for communication. Communication may also take the form of conducted communication in which electrical signals and potential differences therefrom are conducted through the body.

In various embodiments, the electrical sensing circuitry 408 may be configured to detect activity of a body part of the patient. In some examples, the activity may include the cardiac electrical activity of the heart. For example, the electrical sensing circuitry 408 may be connected to electrodes 426A-426F and the electrical sensing circuitry 408 may be configured to receive and measure the cardiac electrical activity of the heart. The electrical sensing circuitry 408 may also be connected to the processing circuitry 412 and provide signals (i.e., data) representative of the cardiac electrical activity to the processing circuitry 412. The electrical sensing circuitry 408 may also be controllable as by, for example, having a controllable sampling rate, frequency band, slew rate, sensitivity and/or dynamic range to accommodate different conditions in the body and/or signal characteristics. The electrical sensing circuitry 408 may include one or more analog-to-digital converter sub-circuits and/or sample/hold circuitry for use in sampling the sensed signal and converting the sensed signal to a form that can be stored in memory 416 and/or processed in the processing circuitry 412. In an example, the electrical sensing circuitry 408 may be integrated into the processing circuitry 412, if desired.

In some examples, the oscillatory sensing circuitry 410 may be configured to detect activity of a body part of the patient. In some examples, the activity may include, but is not limited to, the motion of the heart, breathing of a lung or lungs, and motion of the patient's muscles, bones, other internal tissue, etc. In certain embodiments, the oscillatory sensing circuitry 410 may be connected to oscillatory sensors 428A, 428B. According to various embodiments, the oscillatory sensors 428A, 428B may include, but are not limited to vibrational sensors, accelerometers, pressure sensors, displacement sensors, velocity sensors, strain sensors, heart sound sensors, microphones, hydrophones, blood-oxygen sensors, chemical sensors, temperature sensors, flow sensors and/or any other suitable sensors that are configured to detect one or more activities of the body and/or the body part of the patient (e.g., heart motion, heart sound, breathing of the lung, muscle movement, etc.). In some cases, the oscillatory sensors 428A, 428B may provide signals (i.e. data) representative of the activity to the oscillatory sensing circuitry 410 and the oscillatory sensing circuitry 410 may receive and measure the received signals. The oscillatory sensing circuitry 410 may also be connected to the processing circuitry 412 and provide signals representative of the measurements to the processing circuitry 412. The oscillatory sensing circuitry 410 may also be controllable as by, for example, having a controllable sampling rate, frequency band, slew rate, sensitivity and/or dynamic range to accommodate different conditions in the body and/or signal characteristics. The oscillatory sensing circuitry 410 may include one or more analog-to-digital converter sub-circuits and/or sample/hold circuitry for use in sampling the sensed signal and converting the sensed signal to a form that can be stored in memory 416 and/or processed in the processing circuitry 412. Although described with respect to FIG. 4 as separate sensing circuitries, in some cases, the electrical sensing circuitry 408 and the oscillatory sensing circuitry 410 may be combined into a single sensing circuitry, as desired. Furthermore, in some examples, the electrical sensing circuitry 408, the oscillatory sensing circuitry 410, and the processing circuitry 412 may be combined into a single circuitry, if desired.

When so provided, the pulse generator circuitry 418 may be configured to generate electrical pulses. For example, the pulse generator circuitry 418 may generate and deliver electrical pulses by using energy stored in the power source 416 and deliver the generated pacing pulses via the electrodes 426A-426F and/or sensors 428A, 428B. Alternatively, or additionally, the pulse generator circuitry 418 may include one or more capacitors, and the pulse generator circuitry 418 may charge the one or more capacitors by drawing energy from the power source 416. The pulse generator circuitry 418 may then use the energy of the one or more capacitors to deliver the generated pacing pulses via the electrodes 426A-426F, and/or sensors 428A, 428B. In at least some examples, the pulse generator circuitry 418 may include switching circuitry to selectively connect one or more of the electrodes 426A-426F and/or sensors 428A, 428B to the pulse generator circuitry 418 in order to select which of the electrodes 426A-426F and/or sensors 428A, 428B (and/or other electrodes) the pulse generator circuitry 418 uses to deliver the electrical pulses and/or stimulation therapy. An H-Bridge is one known circuit for therapy output control.

The pulse generator circuitry 418 may generate and deliver electrical stimulation signals with particular features or in particular sequences in order to provide one or multiple of a number of different stimulation therapies. For example, the pulse generator circuitry 418 may be configured to generate electrical stimulation signals to provide electrical stimulation therapy to combat bradycardia, tachyarrhythmias, atrial or ventricular fibrillation and/or to produce any other suitable electrical stimulation therapy such as cardiac resynchronization therapy (CRT). Some more common electrical stimulation therapies include bradycardia pacing therapy, anti-tachycardia pacing (ATP) therapy, CRT, and cardioversion/defibrillation therapy.

The pulse generator circuitry 418 may also be configured to deliver pulses at two or more different energy levels and/or at energy levels that can be configured by a physician or user. This may be accomplished by controlling the pulse frequency, slew, width, pulse intensity, pulse shape or morphology, and/or any other suitable pulse characteristic. Moreover, in some cases, the pulse generator circuitry 418 may allow the processing circuitry 412 to control the pulse frequency, slew, width, pulse intensity, pulse shape or morphology, and/or any other suitable pulse characteristic.

The processing circuitry 412 may include electronics that are configured to control the operation of the IMD 400. For example, the processing circuitry 412 may be configured to receive electrical signals from the electrical sensing circuitry 408 and/or the oscillatory sensing circuitry 410. Based on the received signals, the processing circuitry 412 may obtain, for example, a physiological parameter(s) of a body part and monitor the status of the physiological parameter. The processing circuitry 412 may then control the telemetry circuitry 406 to send a communication signal indicative of the status of the physiological parameter to an external device. The processing circuitry 412 may further receive information from the telemetry circuitry 406. In some examples, the processing circuitry 412 may use such received information to help detect the activity of a body part, monitor physiological parameters from the activity, determine whether an abnormality is occurring or has changed, and take a particular action in response to the information.

In some examples, the processing circuitry 412 may include a pre-programmed chip, such as a very-large-scale integration (VLSI) chip and/or an application specific integrated circuit (ASIC). In such embodiments, the chip may be pre-programmed with control logic in order to control the operation of the IMD 400. For example, a state machine architecture may be used. By using a pre-programmed chip, the processing circuitry 412 may use less power than other programmable circuits (e.g. general purpose programmable microprocessors) while still being able to maintain basic functionality, thereby potentially increasing the battery life of the IMD 400.

In other examples, the processing circuitry 412 may include a programmable microprocessor. Such a programmable microprocessor may allow a user to modify the control logic of the IMD 400 even after implantation, thereby allowing for greater flexibility of the IMD 400 than when using a pre-programmed ASIC. In some examples, the processing circuitry 412 may store information on and read information from the memory 416. In other examples, the IMD 400 may include a separate memory (not shown) that is in communication with the processing circuitry 412, such that the processing circuitry 412 may read and write information to and from the separate memory.

According to various embodiments, the physiological parameters obtained by the processing circuitry 412 may be indicative of the state of the body part of the patient and/or the patient themselves. For example, in some cases, the physiological parameter may be a heart sound that includes sounds caused by the motion of the heart including heart sounds S₁, S₂, S₃, S₄, cardiac murmurs, cannon waves, cannon sounds, etc. Furthermore, in various examples, the status and/or change in status of the physiological parameter(s) may indicate abnormalities of the body part and/or the patient themselves. For example, the status and/or change in status of a respective physiological parameter may indicate mitral valve regurgitation, tricuspid valve regurgitation, atrial fibrillation, valvular stenosis, etc.

Accordingly, in an illustrative example, the oscillatory sensor 428A may be located in a right ITV of the patient and the oscillatory sensor 428B may be located in a left ITV of the patient. In this example, the oscillatory sensors 428A, 428B may be accelerometers and configured to detect the motion of the heart and provide signals representative of the motion of the heart to the oscillatory sensing circuitry 410. The oscillatory sensing circuitry 410, in turn, may measure the received signals and provide signals representative of the measurements to the processing circuitry 412.

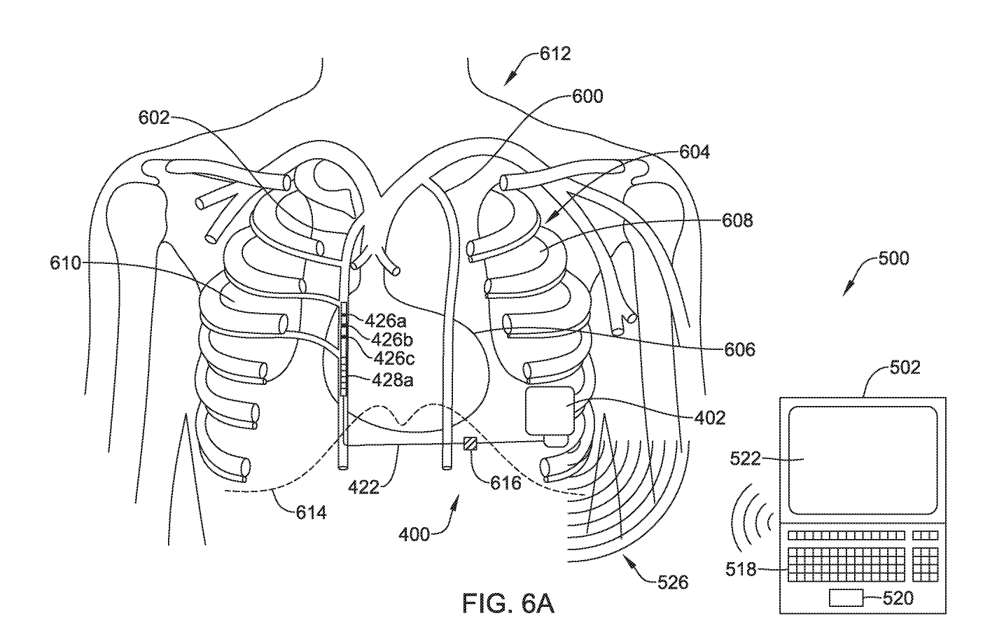

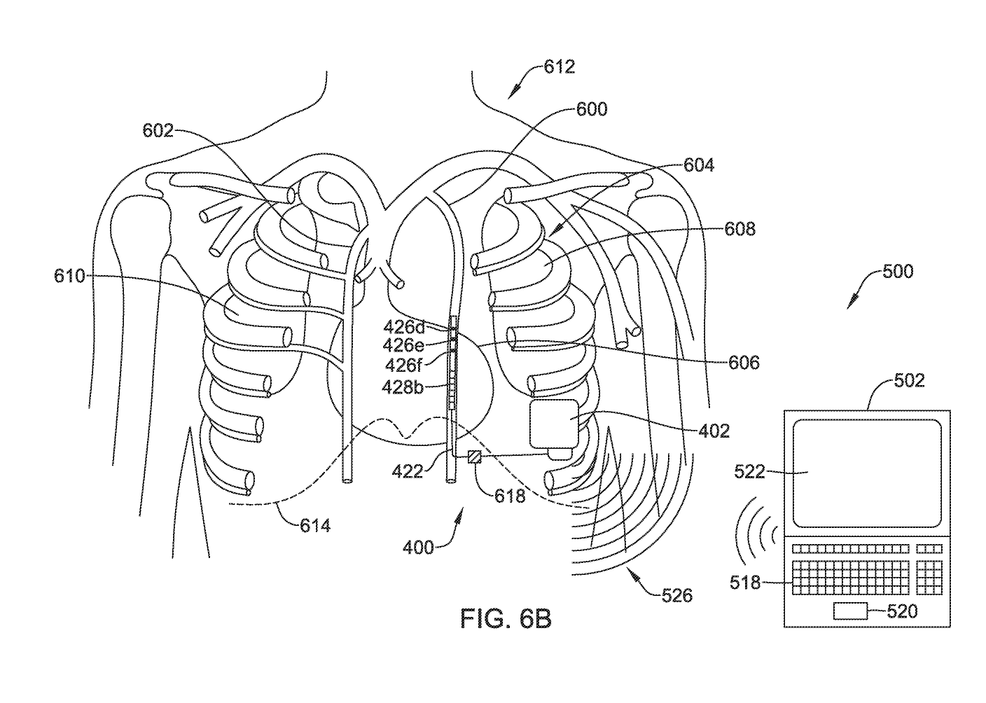

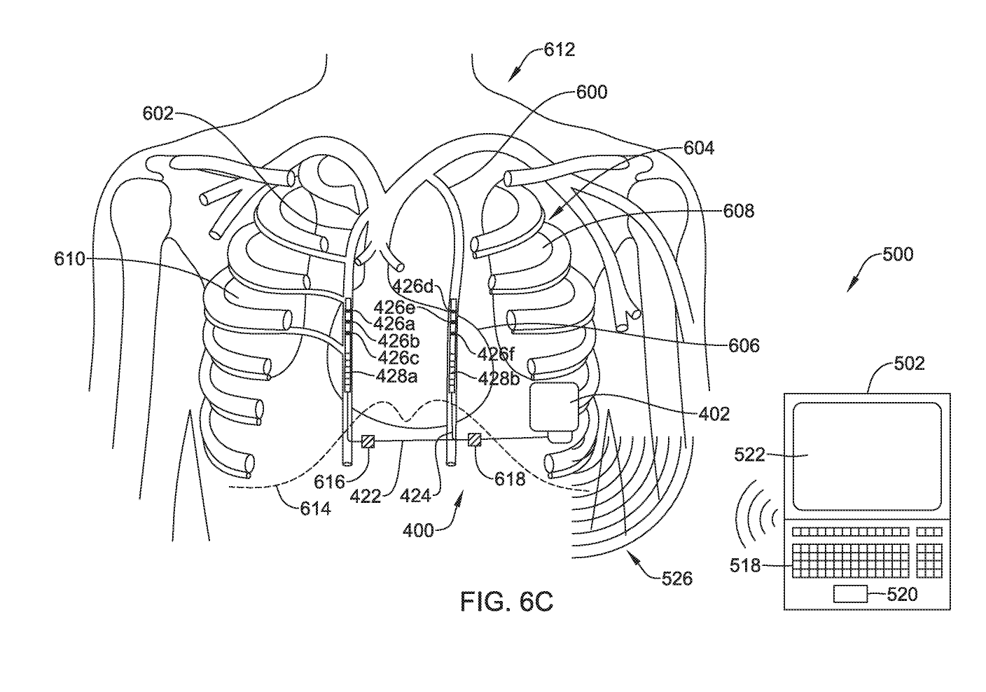

In an example, the processing circuitry may use signals from one or more of the electrodes 426A-426D to sense a cardiac cycle in order to set windows for detection of motion or sound activity of the heart using the sensors 428A, 428B. The example of FIG. 4 indicates the inclusion of two oscillatory sensors 428A, 428B, however, in other examples, such as shown in FIGS. 6A-6B, below, a single oscillatory sensor 428A, 428B may be used.

From the signals, the processing circuitry 412 may obtain and monitor the status of the heartbeat. From monitoring the heartbeat, the processing circuitry 412 may detect an occurrence of a heart sound, such as one or more of S₁, S₂, S₃ and S₄, as those terms are known in the art, or alternatively a sound indicating potential abnormality such as a cannon sound or a heart murmur. Such sensing may be used to then determine whether an indication or alert is needed and, if so, to generate the indication or alert. For example, the IMD 400 may include a beeper and/or a vibration mechanism (not shown) and the indication or alert may be generated by activating the beeper and/or vibration mechanism.

In another example, in some cases, the occurrence of a heart murmur may not have been detected before for a given patient. In other cases, there may have already been a heart murmur known to exist, however, the processing circuitry 412 may detect a change in the heart murmur by, for example, comparing a sensed parameter associated with the known heart murmur to a baseline for the parameter, where the parameter may be, for example, a frequency band, an amplitude or intensity, a waveform shape, timing relative to one or more other events in the cardiac electrical or other signal (such as timing relative to the electrical P-wave or R-wave or timing relative to one of S1, S2, S3, S4).

If an alert is needed, the device may comprise annunciating circuitry to generate a vibration or a tone/sound to the user itself, or may communicate using the telemetry circuitry 406 to communicate the occurrence and/or change of the heart murmur to an external device. In this example, the external device may be the patient's mobile device such as a phone or watch having Bluetooth or other communications capability through the telemetry circuitry 406. The mobile device may then present an indication of the occurrence or change of the heart murmur to the patient. For instance, the mobile device may include a user-interface with illuminating devices such as LED's, or audio devices, such as speakers or buzzers, to provide the indication. For example, a heart murmur indication may be displayed using a red LED. In this case, the patient may observe that the red LED has turned on and may notify their physician. The external device may be on a cellular or other network allowing further annunciation directly to the patient's physician or to a “call” or “message” center for receiving such communications from external devices associated with implantable devices.

In further embodiments, the housing 402 may also include a sensor (not shown). Continuing with the heart murmer example, in response to detecting the heart murmur and/or change in the heart murmur, the processing circuitry 412 may select the sensors 428A, 428B and the housing 402 sensor to triangulate a source of the heart murmur. Once the source has been located, the processing circuitry 412 may use that information to associate or determine if the heart murmur is due to a cardiac abnormality (e.g., mitral valve regurgitation, tricuspid valve regurgitation, etc.). In this example, the processing circuitry may associate the heart murmur with mitral valve regurgitation. The processing circuitry 412 may then use the telemetry circuitry 406 to communicate the occurrence of the heart murmur due to mitral valve regurgitation to the mobile device. The mobile device may then present the indication of the occurrence of the heart murmur due to mitral valve regurgitation by turning on the red LED and use the speakers to produce an “alert” sound to the patient. In this case, the patient may understand that the combination of the red LED turning on and the alert sounding signifies an emergency event and use the mobile device to send a communication signal to the telemetry circuitry 406 with instructions to administer cardiac therapy. The telemetry circuitry 406 may relay the information to the processing circuitry 412 and the processing circuitry 412 may use the pulse generator circuitry 418 to administer cardiac stimulation therapy to the heart. In other embodiments, the processing circuitry 412 may automatically use the pulse generator circuitry 418 to administer cardiac stimulation therapy to the heart without receiving instructions from the mobile device, given the severity of the situation.

In some examples, after the pulse generator circuitry 418 has administered cardiac stimulation therapy, the processing circuitry 412 may use the electrical sensing circuitry 408 and/or the oscillatory sensing circuitry 410 to determine if the cardiac therapy has achieved adequate results. In some cases, if the cardiac therapy did not work, the processing generator circuitry 418 may modify the pacing settings of the pulse generator circuitry 418 by increasing or decreasing the pace amplitudes or changing the timing to deliver the cardiac therapy pulses at different intervals. The processing circuitry 412 may then use the pulse generator circuitry 418 to administer the cardia stimulation therapy again and determine if the cardiac therapy achieved adequate results.

In some examples, from monitoring the physiological parameter(s) (e.g., the heartbeat), the processing circuitry 412 may observe erratic heart sound(s) and observe very fast R-waves or disorganized electrical signals. As a result, the processing circuitry 412 may determine and/or confirm that the heart is experiencing arrhythmia. Accordingly, the processing circuitry 412 may use the pulse generator circuitry 418 to administer cardiac stimulation therapy to the heart. Thus, for example, fast R-waves or disorganized cardiac electrical signals may be observed using a monitored cardiac electrical signal, and the oscillatory sensor may be used to aid in differentiating a fast ventricular rhythm which is not harmful (such as exercise induced ventricular tachycardia) from one that is (such as ventricular fibrillation)—in the former, a regular heart sound signal would be expected, though at elevated rate, while in the latter, the heart sound signal would become irregular as the pattern of valve closing and opening sounds would change. A cardiac therapy may be delivered in response to finding that the oscillatory sensor indicates an unusual, irregular, or otherwise unhealthy state of body part activity. In some examples, delivery of therapy such as a cardiac electrical therapy (or a drug therapy or other electrical therapy, for example) may be determined at least in part by the use of the oscillatory signal representing activity of the heart, lung(s) or other body organ.

In some examples, from monitoring the physiological parameter(s) (e.g., the heartbeat), the processing circuitry 412 may observe that one or more heart sound(s) becomes weak while others occur at a fast rate, indicating a ventricular originating tachycardia (i.e. the heart is not properly filling between beats) that may be pace terminated. The processing circuitry 412 may then observe that the electrical signals are regular but fast and wide, confirming that the tachycardia is pace terminable. As a result, the processing circuitry 412 may use the pulse generator circuitry 418 to administer antitachycardia pacing (ATP) to the heart.

In some examples, from monitoring the physiological parameter(s) (e.g., the heartbeat), the processing circuitry 412 may observe heart sounds that have erratic timing, indicating varying interbeat intervals. The processing circuitry 412 may then determine the heart is experiencing atrial fibrillation affecting ventricular rate. However, the processing circuitry 412 may then observe that the heart sounds themselves are essentially normal, suggesting that the patient remains asyptomatic. As a result, the processing circuitry 412 may determine that therapy is not needed for the AF. If the processing circuitry 412, at any time, were to observe the heart sounds breaking down, the processing circuitry 412 may then send an “alert” for the AF condition and/or deliver therapy to cardiovert the atria.

In some examples, from monitoring the physiological parameter(s) (e.g., the heartbeat), the processing circuitry 412 may observe the heart sounds remain generally normal, but the beat rate increases greatly. In response, the processing circuitry 412 may use the oscillatory sensing circuitry 410 to detect and/or perform gait analysis. In some embodiments, the processing circuitry 412 may adjust a parameter set of the oscillatory sensing circuitry 410 and monitor the activity level of the patient. In some cases, from the activity level, the processing circuitry 412 may determine that the high beat rate is due to exercise induced sinus tachycardia. As a result, the processing circuitry 412 may determine that therapy is not needed.

In some examples, from monitoring the physiological parameter(s), the processing circuitry 412 may determine that the electrical sensing circuitry 408 and/or the oscillatory sensing circuitry 410 may not be obtaining correct results and/or are not functioning properly. In this case, the processing circuitry 412 may modify the sensing settings of the IMD 400 to obtain accurate results.

In other cardiac examples, other elements of captured signals may be monitored. For example, heart sounds (S1, S2, S3, S4) may be monitored using the oscillatory sensor and compared to stored templates or baseline parameters, or even to a dynamic parameter as a recent trend of a parameter, to observe outlier behavior indicating a change in cardiac status. For example, a time delay between one of the heart sounds and a delivered CRT pulse may be monitored to determine whether the CRT pulse has been delivered a desired time of the cardiac cycle, or time delay between the CRT pulse(s) and a heart sound may be monitored, or the frequency content, amplitude or other feature of the heart sound may be monitored for changes, to indicate whether CRT is having a desired effect.

In some cases, the detected body part may be the lung and the physiological parameter may include respiratory interval, respiratory amplitude, respiratory sound, respiratory rate, respiratory depth, a frequency of oscillation, a pattern of oscillation, etc. Furthermore, in various examples, the status and/or change in status of the physiological parameter(s) may indicate abnormalities of the lung and/or the patient themselves. For example, the status and/or change in status of a respective physiological parameter may indicate wheezing, rales, snoring, rhonchi, a pathological asymmetrical respiratory pattern, asthma or a change in the patient's asthma, Chronic Obstructive Pulmonary Disease (COPD) or a change in the patient's COPD, pneumonia or a change in the patient's pneumonia, etc.

Accordingly, in an illustrative example, the oscillatory sensor 428A may once again be located in the right ITV of the patient and the oscillatory sensor 428B may be located in the left ITV of the patient. In this example, the oscillatory sensors 428A, 428B may be accelerometers and configured to detect the breathing of the lung and provide signals representative of the breathing to the oscillatory sensing circuitry 410. The oscillatory sensing circuitry 410, in turn, may measure the received signals and provide signals representative of the measurements to the processing circuitry 412. From the measurements, the processing circuitry 412 may obtain and monitor the respiratory interval of the breathing. From monitoring the respiratory interval, the processing circuitry 412 may detect a respiratory distress such as asthma, COPD, pneumonia, for example, in the patient. In this case COPD may be detected. In some cases, the observation of COPD may not have been detected before from the respiratory interval. In other cases, the patient may have already been diagnosed with COPD, however, the processing circuitry 412 may observe a change in the patient's COPD from the detected respiratory interval. As such, the device itself may generate an alert by vibrating or issuing tones, or the processing circuitry 412 may use the telemetry circuitry 406 to communicate the change in or occurrence of COPD to an external device (e.g., mobile device). The mobile device may then present an indication of the change in or occurrence of COPD to the patient by turning on a red LED or generating a sound or other alert, or the mobile device itself may issue a further communication via the interne or a cellular connection to contact a physician or a remote monitoring center. In this case, the patient may observe that the red LED has turned on and may notify their physician.

According to various embodiments, the power source 414 may provide power to the IMD 400 for its operations. In some instances, the power source 414 may be a rechargeable battery, which may help increase the useable lifespan of the IMD 400. If the power source 414 is rechargeable, additional circuitry to receive power for recharging (such as an inductive coil) may be provided along with charging control circuits and/or safety circuitry known in the art. In still other examples, the power source 414 may be some other type of power source such as a primary cell battery, as desired. Any suitable chemistry for implantable primary cell or rechargeable batteries may be used.

The components of the operational circuitry 404 may comprise suitable sub-circuits such as ASIC or discrete chips for use in telemetry operations, digital and analog logic and/or, if desired, a digital signal processor of the sensing circuitry, as well as suitable pulse frequency controlling circuitry in block 412, which may include its own frequency/oscillating circuitry or may rely upon signals from other components of the operational circuitry.

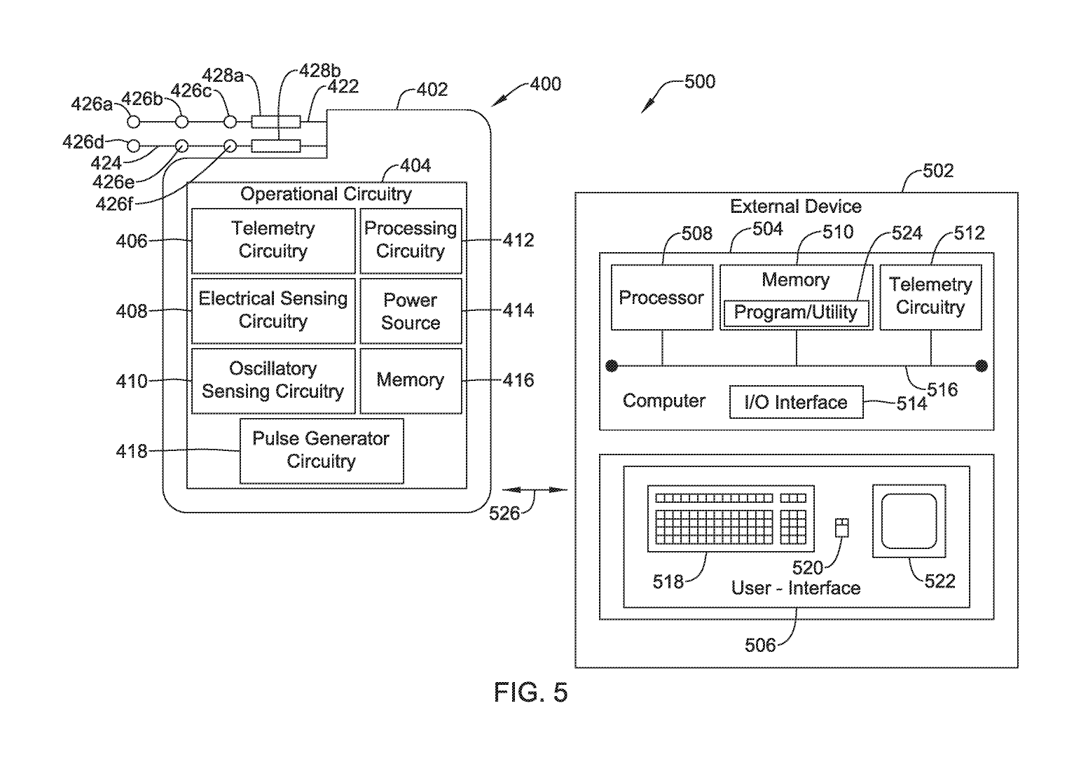

FIG. 5 depicts an exemplary system 500 comprising the IMD 400 and an external device 502 that may be used to treat a patient. According to various embodiments, the external device 502 is of a type that is suitable for accessing and/or utilizing the IMD 400, consistent with embodiments of the present disclosure. The external device 502 provides only an illustration of one implementation and does not imply any limitations with regard to the environments in which different embodiments may be implemented. Many modifications to the external device 502 may be made based on design and implementation requirements. Examples of external devices, environments, and/or configurations that may be represented by the external device 502 include, but are not limited to, desktop computers, laptop computers, tablet computers, smartphones, server computers, thin clients, thick clients, multiprocessor systems, microprocessor-based systems, and distributed cloud computing environments. In some cases, the external device 502 merely provides a user interface for an installer or the like to interact with the IMD 400.

In certain embodiments, components of the external device 502 may include a computer 504 and a user interface 506. Components of the computer 504 may include a processor 508, a memory 510, telemetry circuitry 512, and an I/O interface 514. Each of the components of the operational circuitry 504 may be connected to an internal bus 516 that includes data, address, and control buses, to allow the components of the computer 504 to communicate with each other via the bus 516.

In certain embodiments, the processor 508 may be a central processing unit (CPU) that executes an operating system and computer software executing under the operating system. In some cases, the processor 508 may also execute other instructions stored in the memory 510. The memory 510 can include computer system readable media in the form of volatile memory, such as random access memory (RAM) and/or cache memory. The memory 510 may further include other removable/non-removable, volatile/non-volatile computer system storage media. By way of example only, the memory 510 may include a storage system for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus 516 by one or more data media interfaces. As will be further depicted and described below, the memory 510 may include at least one program product having a set of program modules that are configured to carry out the functions of providing instructions to the IMD 400.

In one example, program/utility 524 may be stored in the memory 510 and may include a set of application program modules (e.g. software). In some cases, the program/utility 524 may also include an operating system and program data. According to various embodiments, the application program modules may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

In various embodiments, the computer 504 may communicate with one or more external devices such as the user interface 506. In some cases, the user interface 506 may include a keyboard 518, a touchpad 520, and a display 522, which enable a user to interact with the operational circuitry 504 via the I/O interface 514. A touchscreen may be provided, combining for example the keyboard 518, the touchpad 520 and the display 522 together.

As stated herein, in various embodiments, the IMD 400 may communicate with one or more devices such as the external device 502. Such communication 526 can occur via the telemetry circuitry 406 of the IMD 400 and the telemetry circuitry 512 of the external device 502. The telemetry circuitry 512 may be internal to the external device 502 or may, in some examples, be provided as a wand or dongle that plugs into a port such as USB port of the external device.

For example, in some cases, the display 522 may visually display an application program from the program/utility 524 that enables a user (e.g., a physician) to detect activity of a body part of a patient. The physician may then use the touchpad 520 and/or the keyboard 518 to select the body part and/or region of the body part (e.g., the heart, the lungs, the right atrium of the heart, the left lung, etc.) for which to detect the activity, the activity to detect, and the physiological parameter(s) to monitor from the activity. The application program instructions may then be sent to the processor 508. The processor 508 may then use the telemetry circuitry 512 to communicate 526 the instructions to the telemetry circuitry 406 of the IMD 400. The telemetry circuitry 406 may then relay the instructions to the processing circuitry 412. The instructions may inform the processing circuitry 412 to use the oscillatory sensing circuitry 410 to detect the specified activity of the specified body part or body part region using the appropriate oscillatory sensors 428A, 428B. In another embodiment, the instructions may inform the processing circuitry 412 to use the electrical sensing circuitry 408 to detect the specified activity of the specified body part or body part region using the appropriate electrodes 426A-426F. In response, the oscillatory sensors 428A, 428B may detect the specified activity of the specified body part or body part region and provide signals representative of the activity to the oscillatory sensing circuitry 410. The oscillatory sensing circuitry 410, in turn, may measure the received signals and provide signals representative of the measurements to the processing circuitry 412. From the signals, the processing circuitry 412 may obtain and monitor the status of the specified physiological parameter(s). As such, the processing circuitry 412 may use the telemetry circuitry 406 to communicate 526 the status of the physiological parameter(s) to the telemetry circuitry 512 of the external device 502. The telemetry circuitry 512 may then relay the status of the physiological parameter(s) to the processor 508. The processor 508 may then use the display 522 to present an indication of the status of the physiological parameter(s) to the physician.

FIG. 6A depicts a first configuration of the system 500 implanted in a patient 612. FIG. 6A illustrates portions of the thoracic anatomy of the patient 612 including location of the left ITV 600 and the right ITV 602. The ribcage is shown at 604, an outline of the heart is shown at 606, an outline of the left lung is shown at 608, and an outline of the right lung is shown at 610. The system 500 is also shown having the IMD 400 (e.g., an ICD device) with the lead 422 located in the right ITV 602 of the patient. In certain embodiments, as shown, the lead 422 may include the electrodes 426A-426C and the oscillatory sensor 428A spaced from one another. According to various embodiments, the distal portion of the lead 422 may include a fixation apparatus or shape for the flexible lead, such as a 2 or 3 dimensional curve, tines, an expandable member, hooks, a side-extending engagement structure, etc. A number of examples for lead shape and design for implantation using the ITV may be found in U.S. patent application Ser. No. 15/846,060, title LEAD WITH INTEGRATED ELECTRODES, U.S. Provisional Patent Application No. 62/452,537, title IMPLANTABLE MEDICAL DEVICE, and U.S. Provisional Patent Application No. 62/486,635, titled ACTIVE MEDICAL DEVICE WITH ATTACHMENT FEATURES, the disclosures of which are incorporated herein by reference.

Access to the right ITV 602 may be achieved at any location, such as superior or inferior positions. FIG. 6A shows implantation from an inferior position in the right ITV 602. In this example, the right ITV 602 has been accessed by introduction through its respective superior epigastric veins from a location inferior to the rib margin 614. The housing 402 of the IMD 400 has been placed at approximately the left axilla. The housing 402 may be placed as desired, for example at the anterior axillary line, the midaxillary line, or in the posterior axillary line.

In the illustration, a suture sleeve is shown at 616 and is used to fixate the lead 422 for example, to the subcutaneous fascia. For placement of the lead 422, the right ITV 602 may be accessed and a tunnel established between the left axilla and the access location such as along a portion of the inframammary crease. The lead 422 may, in this case, be relatively stiff to assist in keeping it emplaced in the patient as shown, if desired.

In the example of FIG. 6A, a left axillary housing location is shown; a right sided, pectoral or subclavicular left or right position may be used instead, in combination with the right ITV 602 placement and/or the left ITV 600 placement. Illustrative examples of additional implant locations are shown in U.S. patent application Ser. No. 15/846,081, titled IMPLANTATION OF AN ACTIVE MEDICAL DEVICE USING THE INTERCOSTAL VEIN and U.S. patent application Ser. No. 15/868,799, titled IMPLANTATION OF AN ACTIVE MEDICAL DEVICE USING THE INTERNAL THORACIC VASCULATURE, the disclosures of which are incorporated herein by reference.

The ITV's 600, 602 may be accessed via their corresponding superior epigastric veins. For example, access may be achieved using ultrasound guided needle insertion. The access method may resemble the Seldinger technique. Other venipuncture or cutdown techniques may be used instead.

The Seldinger technique may include creating a puncture at the desired access location, with a hollow needle or trocar, for example under ultrasound guidance, introducing a guidewire through the needle and into the desired blood vessel, removing the needle, keeping the guidewire in place, and then inserting an introducer sheath, which may have a valve at its proximal end, over the guidewire. The introducer sheath may be advanced to a location to place its distal tip near a desired location. Contrast injection may be useful to visualize the superior epigastric vein and/or ITV structures. A guide catheter and guidewire may then be introduced through the introducer sheath. The guidewire may be the same as used in gaining initial access (if one is used to gain access), or may be a different guidewire. In another example, a cut-down technique may be used to access the desired superior epigastric vein by incision through the skin. The incision may be made laterally from the location of the desired vein. Next, possibly after visual confirmation the desired vessel is accessed, incision into the selected vein can be made, followed by insertion of the lead. Once access to the right superior epigastric vein is achieved, the vessel can be traversed in a superior direction to place the lead 422 with the electrodes 426A-426C at the desired level by entering the right ITV 602.

Various approaches for use of the ITV are shown in U.S. Provisional patent application Ser. No. 15/801,719, titled PARASTERNAL PLACEMENT OF AN ACTIVE MEDICAL DEVICE USING THE INTERNAL THORACIC VASCULATURE, U.S. patent application Ser. No. 15/814,990, titled TRANSVENOUS MEDIASTINUM ACCESS FOR THE PLACEMENT OF CARDIAC PACING AND DEFIBRILLATION ELECTRODES, and U.S. Provisional Patent Application No. 62/473,882, titled IMPLANTABLE MEDICAL DEVICE, the disclosures of which are herein incorporated by reference.

The lead 422 may be tunneled from the parasternal access location across and down to the housing 402, which may be implanted at the left axilla as illustrated. For ease of illustration the housing 402 is shown at about the anterior axillary line, level with the cardiac apex and/or inframammary crease. In other examples the housing 402 may be more lateral and/or posterior, such as at the mid-axillary line or posterior axillary line, or may even be more dorsal with placement dorsally between the anterior surface of the serratus and the posterior surface of the latissimus dorsi. A right sided axillary, pectoral or subclavicular left or right position may be used instead, in combination with right or left ITV placement.

In some examples, a flexible lead may be introduced with the support of a guide catheter during advancement. The guide catheter may receive the lead through a guide catheter lumen that serves to retain a fixation apparatus or shape for the flexible lead, such as a 2-dimensional or 3-dimensional curvature, tines, an expandable member, or hooks or a side-extending engagement structure. A stylet may be placed through the lead, or a portion thereof, to retain a straight shape during implantation; upon removal of the stylet, a curvature may then be released for securing the lead in place.

In another alternative, the guide catheter and guidewire may be omitted by providing a lead with a flexible or steerable structure, and/or a lead configured for implantation using a steerable stylet. For example, a lead may be configured to be implanted using a steerable stylet in a lumen thereof, with the initial placement into the right ITV 602 (or left ITV 600, if desired) at the distal end of the introducer sheath, possibly using contrast visualization, if desired. Once initial access is achieved, simply pushing the stylet should be sufficient to implant the lead to a desired location in the ITV. The stylet may have a secondary function of preventing an anchoring structure of the lead from assuming an anchoring shape or releasing an anchoring tine, hook, expandable member, stent or other device. In other examples, a guidewire and/or sheath may not be needed. Due to the limited angulation required for accessing the ITV from a parasternal incision, the lead may be inserted directly into the ITV, reducing the time and complexity of the procedure.

The lead 422 shown in FIG. 6A includes the three ring electrodes 426A-426C and the oscillatory sensor 428A. The ring electrodes 426A-426C and oscillatory sensor 428A may serve to sense body parts of the patient 612. The housing 402 may also include an electrode(s) or oscillatory sensor(s) for sensing internal body of the patient 612.

According to various embodiments, in the example shown in FIG. 6A, an application program may be presented on the display 522 of the external device 502 to a user (e.g., a physician). The user may use the touchpad 520 and/or the keyboard 518 to select the body part and/or region of the body part of the patient 612 for which to detect activity, the activity to detect, and the physiological parameter(s) to monitor from the activity. In various embodiments, the physiological parameters may be indicative of the state of the body part of the patient and/or the patient themselves. For example, in some cases, the physiological parameter may be a heart sound that includes sounds caused by the motion of the heart including heart sounds S₁, S₂, S₃, S₄, cardiac murmurs, cannon waves, cannon sounds, etc. Furthermore, in various examples, the status and/or change in status of the physiological parameter(s) may indicate irregularities of the body part and/or complications the patient may be suffering. For example, the status and/or change in status of a respective physiological parameter may indicate that the patient's heart is suffering mitral valve regurgitation, tricuspid valve regurgitation, atrial fibrillation, valvular stenosis, etc. In this example, the physician may choose to monitor the heart sound from the motion of the heart 606.

In some examples, the oscillatory sensor 428A may operate independent of other sensors in the system. In other examples, a second signal, such as a cardiac electrical signal captured using one or more electrodes 426A, 426B, 426C, and/or the housing of the implantable device, may be sensed and used to set windows for observation of the output of the oscillatory sensor 428A.

Based on the instructions from the external device 502, the IMD 400 may select the oscillatory sensor 428A to detect the motion of the heart 606. In this example, the oscillatory sensor 428A may be a microphone. However, in other examples, the oscillatory sensor 428A may be a vibrational sensor, an accelerometer, a pressure sensor, a heart sound sensor, a hydrophone, a blood-oxygen sensor, a chemical sensor, a temperature sensor, a flow sensor and/or any other suitable sensor or combination thereof that are configured to detect one or more activities of the heart. Once the microphone has detected the motion of the heart 606, the microphone may provide signals representative of the motion of the heart to the IMD 400. The IMD 400 may then obtain and monitor the status of the heartbeat.

From monitoring the heartbeat, the IMD 400 may observe many abnormalities such as, to name a few, an occurrence of S₄, which may be caused by atrial kick during diastole, a heart murmur that may be caused by regurgitation or valvular stenosis, or heart sounds that may be related to pulmonary congestion. In this example, the IMD 400 may detect an occurrence of a cannon wave and/or a cannon sound coming from the right atrium during the heartbeat. In some cases, the occurrence of the cannon wave and/or cannon sound may not have been detected before from the heartbeat. In other cases, there may have already been a cannon wave and/or cannon sound in the heartbeat. However, the IMD 400 may detect a change in the cannon wave and/or cannon sound. As such, the IMD 400 may communicate 526 the occurrence of and/or change in the cannon wave and/or cannon sound to the external device 502. In this example, the external device 502 may present an indication of the occurrence of and/or change in the cannon wave and/or cannon sound by displaying a message on the display 522 to the physician.

In various embodiments, the physician may use the application program to perform further observation of the heartbeat by using the touchpad 520 and/or the keyboard 518 to select oscillatory sensors (not shown) (i.e., accelerometers) included on the housing 402 of the IMD 400 to also detect the motion of the heart 606. Based on the instructions from the external device 502, the IMD 400 may select the oscillatory sensor 428A (i.e. the microphone) and the accelerometers to triangulate the cannon wave and/or cannon sound in the heartbeat. Once the cannon waves and/or cannon sounds have been identified, the IMD 400 may use that information to determine the severity of the cannon waves and/or cannon sounds. In some cases, the IMD 400 may associate the severity of the cannon waves and/or cannon sounds with atrial fibrillation (AF). Furthermore, in certain embodiments, the IMD 400 may also calculate the degree of AF. As such, the IMD 400 may communicate 526 the diagnosis to the external device 502 and the external device 502 may display the diagnosis to the physician. In this case, given the severity of the situation, the physician may utilize the application program to administer cardiac stimulation therapy using the electrodes 426A-426C of the IMD 400. In other embodiments, the IMD 400 may automatically select the electrodes 426A-426C and administer cardiac stimulation therapy to the heart 606 without receiving instructions from the external device 502.

In certain embodiments, detection of the activity of the lungs may also provide physiological parameters regarding the overall health of the patient 612. For instance, the status and/or change in the status of the physiological parameters such as respiratory interval, respiratory amplitude, respiratory sound, respiratory depth, respiratory rate, a frequency of oscillation, and/or a pattern of oscillation of the lungs may indicate that the patient 612 may be experiencing wheezing, rales, snoring, rhonchi, a pathological asymmetrical respiratory pattern, asthma or a change in asthma, Chronic Obstructive Pulmonary Disease (COPD) or a change in COPD, pneumonia or a change in pneumonia, etc. In this example, the physician may use the external device 502 to monitor the respiratory amplitude during breathing of the right lung 610.

Based on the instructions from the external device 502, the IMD 400 may select the oscillatory sensor 428A to detect the breathing of the right lung 610. In this example, the oscillatory sensor 428A may be a 3-D accelerometer. In some embodiments, the 3-D accelerometer may have higher frequency-sampling capability (e.g. 500-1000 Hz). In addition, the 3-D accelerometer may be configured for dual-modality sensing (e.g. an inertial and acoustic/hydrophone). In this embodiment, the 3-D accelerometer may detect more acoustic-based sounds such as respiratory distress, for example.

Once the 3-D accelerometer has detected the breathing of the right lung 610, the 3-D accelerometer may provide signals representative of the breathing of the right lung 610 to the IMD 400. The IMD 400 may then obtain and monitor the status of the respiratory amplitude of the right lung 610. From monitoring the respiratory amplitude, the IMD 400 may detect a respiratory distress such as asthma, COPD, pneumonia, for example, in the right lung 610. In this case, asthma may be detected. In some cases, the occurrence of the asthma may not have been detected before in the right lung 610. In other cases, the asthma may have already been detected in the right lung 610. However, the IMD 400 may detect a change in the asthma. As such, the IMD 400 may communicate 526 the occurrence of and/or change in the asthma to the external device 502. In this example, the external device 502 may present an indication of the occurrence of and/or change in the asthma by displaying a message on the display 522 to the physician.

According to various embodiments, the ITVs 422, 424 may also provide an opportunity to monitor physiological parameters that may be indicative of the body position of the patient 612 and/or the body movement of the patient 612. As stated herein, the oscillatory sensors 428A, 428B may include, but are not limited to vibrational sensors, accelerometers, pressure sensors, displacement sensors, velocity sensors, strain sensors, heart sound sensors, microphones, hydrophones, blood-oxygen sensors, chemical sensors, temperature sensors, flow sensors and/or any other suitable sensors that are configured to detect one or more activities of the body and/or the body part of the patient. As such, placement of the oscillatory sensors 428A, 428B in the ITVs 422, 424 may allow the oscillatory sensors 428, 428B to detect the motion and reactionary motion of the internal tissue of the patient 612 due to the body position and/or body movement of the patient.

In some examples, the oscillatory sensor 428A and/or 428B may be placed in the ITV itself. In other examples, one or more such sensors may be advanced into an intercostal vein to place the sensor in a location overlying the tissue of interest. Such placement may be aided by putting the oscillatory sensor 428A and/or 428B at or near a distal tip of the lead 422 such that only a distalmost portion of the lead 422 is placed in the intercostal vein.

In another example, a patient's overall body motion may be analyzed using sensors placed as shown herein. For example, gait analysis is the study of human motion. Accordingly, detection of the activity of the muscles may provide physiological parameters to assess, plan, and treat individuals with conditions affecting their posture, their ability to walk, or their movement in general. For instance, the status and/or change in the status of the physiological parameters such as step length, stride length, cadence, speed, dynamic base, progression line, foot angle, hip angle, and/or squat performance may indicate that the patient 612 may be experiencing hemiplegic gait, diplegic gait, neuropathic gait, myopathic gait, choreiform gait, ataxic gait, parkinsonian gait, sensory gait, etc. In this example, the physician may use the external device 502 to monitor the stride length of the patient 612 by detecting muscle activity as the patient 612 walks. The use of the ITV and/or an intercostal vein may enhance gait analysis by placing the sensor closer to a bony structure, as opposed to placing the sensor, for example, in or on the heart itself, which is not attached to the ribs per se. Thus the signal may more closely reflect patient gait when placed in the ITV or in an intercostal vein.

Based on the instructions from the external device 502, the IMD 400 may select the oscillatory sensor 428A to detect the muscle activity while the patient 612 walks. In this example, the oscillatory sensor 428A may be an accelerometer. Once the accelerometer has detected the muscle activity of the patient 612, the accelerometer may provide signals representative of the muscle activity of the patient 612 to the IMD 400. The IMD 400 may then obtain and monitor the status of the stride length of the patient 612. From monitoring the stride length, the IMD 400 may detect an occurrence of parkinsonian gait. In some cases, the occurrence of the parkinsonian gait may not have been detected before in the patient 612. In other cases, the parkinsonian gait may have already been detected in the patient 612. However, the IMD 400 may detect a change in the parkinsonian gait. As such, the IMD 400 may communicate 526 the occurrence of and/or change in the parkinsonian gait to the external device 502. In this example, the external device 502 may present an indication of the occurrence of and/or change in the parkinsonian gait by displaying a message on the display 522 to the physician.

In some examples, the IMD 400 may be in further communication with several external devices. In some cases, one or more of the external devices may be a vagus nerve stimulator. In these cases, the vagus nerve stimulator may be used to change the treatment operation of the IMD 400. For example, in situations where abnormality signals change, such as, COPD, pneumonia, heat failure, etc., the vagus nerve stimulator may adjust the sympathetic tone detection of the IMD 400. In further examples, the vagus nerve stimulator may be in communication with other medical devices implanted within the patient 612. In these cases, the vagus nerve stimulator may implement treatment using these medical devices and/or change the treatment operation of these medical devices. For example, upon receiving the status of the physiological parameter(s) from the IMD 400, the vagus nerve stimulator may instruct a leadless pacemaker located in the heart 606 of the patient 612 to change CRT, bradycardia pacing, initiate ATP, etc. In another example, the vagus nerve stimulator may instruct a DBS system to change Parkinson or other tremor therapy specific to the gait analysis.

FIG. 6B depicts a second configuration of the system 500 implanted in the patient 612. In this configuration the lead 424 is located in the left ITV 600 and a suture sleeve 618 is used to fixate the lead 424 for example, to the subcutaneous fascia. According to various embodiments, the system 500 may operate similar to the operation described in regard to FIG. 6A. However, because of the proximity the lead 424 in the left ITV 600 to certain body parts (e.g., the left lung 608) and/or body part regions (e.g., the left atrium, the left ventricle, etc.), detection and monitoring of these body parts and/or body part regions may be improved.

FIG. 6C depicts a third configuration of the system 500 implanted in the patient 612. In this configuration the lead 422 is located in the right ITV 602 and the lead 424 is located in the left ITV 600. According to various embodiments, the system 500 may operate similar to the operation described in regard to FIG. 6A. However, in this configuration, the detection and monitoring of body parts and/or body part regions of the patient 612 may be improved due to the increased number of electrodes 426A-426F and oscillatory sensors 428A, 428B implanted in the patient 612. For example, implantation of the oscillatory sensor 428A in the right ITV 602 and implantation of the oscillatory sensor 428B in the left ITV 600 may allow for monitoring and diagnosing conditions related to asymmetrical respiratory function.

The configurations depicted in FIGS. 6A-6C provide only three exemplary implementation configurations and do not imply any limitations with regard to configurations in which different embodiments may be implemented. Many modifications to the configurations may be made based on design and implementation requirements. Specifically, implantation configurations of electrodes and oscillatory sensors in the ITV's are all considered and alternate configurations may be dependent upon providing the optimum electrode and oscillatory sensor configuration for detecting and monitoring body parts and/or body part regions of the patient 612.

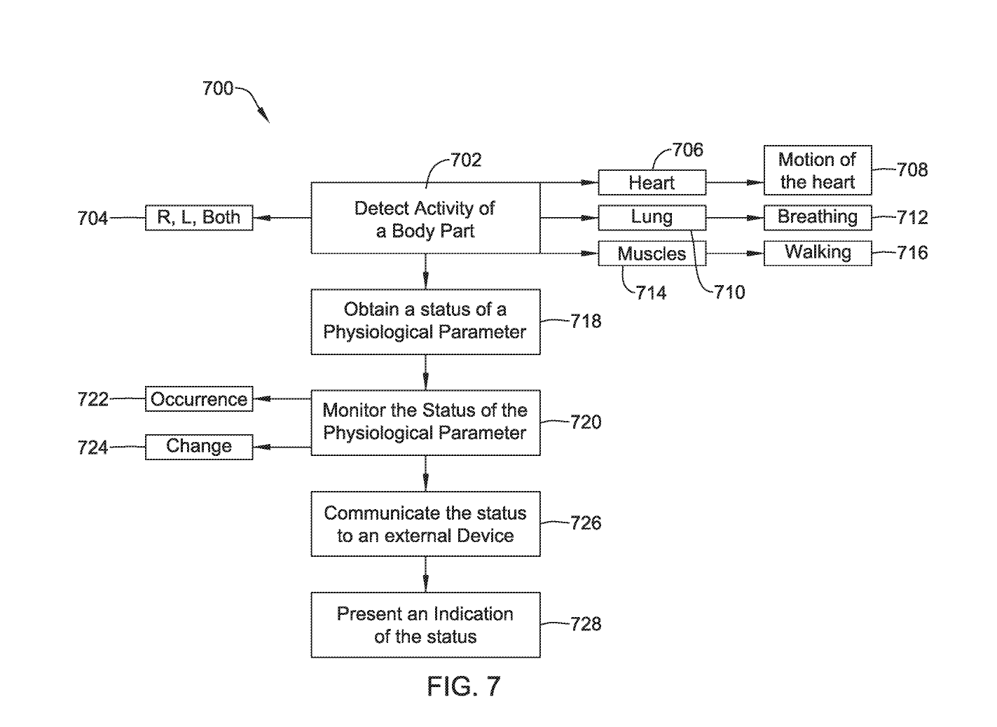

FIG. 7 is a block flow diagram for an illustrative method of treating a patient using an oscillatory sensor. As shown at 700, the method comprises detecting activity of a body part 702, obtaining a status of a physiological parameter 716, monitoring the status of the physiological parameter 720, communicating the status to an external device 726, and presenting an indication of the status 728.

For example, in some embodiments, the oscillatory sensor may be disposed on a lead of an IMD and the lead may be located in an ITV of the patient. In the example, detecting may be done from the right ITV, left ITV, or both ITV, as indicated at 704. Furthermore, the body part detected may be the heart 706 and the activity detected may be the motion of the heart 708. In another example, the body part detected may be a long 710 and the activity detected may be breathing 712. In yet a further example, the body part detected may be the muscles 714 of the patient and the activity detected may be walking 716.

In an example, if the motion of the heart 708 is the activity detected, obtaining a status of a physiological parameter 718 may include obtaining the heart sound that includes sounds caused by the motion of the heart. These sounds may include, but are not limited to heart sounds S₁, S₂, S₃, S₄, cardiac murmurs, cannon waves, cannon sounds, etc. In another example, if breathing 712 is the activity detected, obtaining a status of a physiological parameter 718 may include obtaining respiratory interval, respiratory amplitude, respiratory sound, respiratory depth, respiratory rate, a frequency of oscillation, and/or a pattern of oscillation of the lungs. In yet a further example, if walking 716 is the activity detected, obtaining a status of a physiological parameter 718 may include obtaining step length, stride length, cadence, speed, dynamic base, progression line, foot angle, hip angle, and/or squat performance of the patient.

In an example, monitoring the status of the physiological parameter 720 may include monitoring whether an occurrence 722 of an abnormality can be observed for the physiological parameter. In other examples, the abnormality may have already been observed from the physiological parameter. Monitoring may now include whether a change 724 in the abnormality can be observed from the physiological parameter.

In an example, communicating the status to an external device 726 may include the IMD sending signals representative of the status to the external device. In some examples, the communication may be done via radiofrequency (RF) signals (Bluetooth, ISM, or Medradio, for example), inductive coupling, conducted communication, optical signals, acoustic signals, and/or any other signals suitable for communication.

In an example, presenting an indication of the status 728 may include the external device presenting the indication to a user of the external device, based on the signals sent from the IMD. In some examples, the external device may have a user-interface that includes a display and presenting the indication of the status 728 may include displaying a message on the display to the user. In another example, the user-interface may include illuminating devises such as LED's, or audio devices, such as speakers or buzzers, to present the indication of the status 728 to the user.

Any suitable designs and materials may be used for the leads and electrodes shown and described above. Implantation tools and components may also be of any suitable material and design to allow implantation of the leads and devices described above to the locations discussed.

Some of the examples above may be further characterized as illustrating methods (and devices for performing such methods) for managing a patient, such as by monitoring or providing therapy in response to a patient status or condition, using an implantable medical device (IMD). Such illustrative methods may comprise detecting activity of a body part of the patient using at least one oscillatory sensor of the IMD, the at least one oscillatory sensor located in an internal thoracic vein (ITV) of the patient; determining a status of the body part using the IMD based on the activity of the body part; and monitoring the status of the body part using the IMD by obtaining outputs from the oscillatory sensor. Some examples may further comprise delivering a therapy to the patient with the IMD, wherein the therapy is determined by the IMD using the determined or monitored status of the body part, such as may be done by identifying or confirming presence of a pathological condition such as a deleterious cardiac arrhythmia and delivering anti-tachycardiac pacing, cardioversion, or defibrillation in response thereto. The body part may be the heart or lung of a patient. Both heart and lungs may be monitored with one device, system or sensor, or a plurality of sensors may be provided. The oscillatory sensor may be a microphone or an accelerometer, for example, and the observed activity may be a heart sound or vibration or a lung sound or vibration. Various further details relating to the observed activity are additionally detailed above. The status of the body part may be determined, such as by identifying that the heart is in atrial or ventricular fibrillation, a heart valve is showing signs of poor sealing, or that the heart is behaving a normal, or at least not-unexpected or not-abnormal, state. For the lung, the status of the body part may be, for example, normal, or experiencing abnormal or dysrhythmic behavior, or showing evidence of wheezing, rales, snoring, rhonchi, pathological asymmetrical respiratory pattern, or respiratory distress such as that associated with asthma or COPD. Such body part status may be used to instruct, inform, or modify a therapy such as a delivered drug or electrical therapy, or to generate an alert with an IMD, or to communicate information and/or alerts to an external device in various examples.

The implantable systems shown above may include an implantable pulse generator (IPG) adapted for use in a cardiac therapy system. The IPG may include a hermetically sealed canister that houses the operational circuitry of the system. The operational circuitry may include various elements such as a battery, and one or more of low-power and high-power circuitry. Low-power circuitry may be used for sensing cardiac signals including filtering, amplifying and digitizing sensed data. Low-power circuitry may also be used for certain cardiac therapy outputs such as pacing output, as well as an annunciator, such as a beeper or buzzer, telemetry circuitry for RF, conducted or inductive communication (or, alternatively, infrared, sonic and/or cellular) for use with a non-implanted programmer or communicator. The operational circuitry may also comprise memory and logic circuitry that will typically couple with one another via a control module which may include a controller or processor. High power circuitry such as high power capacitors, a charger, and an output circuit such as an H-bridge having high power switches may also be provided for delivering, for example, defibrillation therapy. Other circuitry and actuators may be included such as an accelerometer or thermistor to detected changes in patient position or temperature for various purposes, and/or output actuators for delivering a drug, insulin or insulin replacement, for example.

Some illustrative examples for hardware, leads and the like for implantable defibrillators may be found in commercially available systems such as the Boston Scientific Teligen™ ICD and Emblem S-ICD™ System, Medtronic Concerto™ and Virtuoso™ systems, and St. Jude Medical Promote™ RF and Current™ RF systems, as well as the leads provided for use with such systems.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method of managing a patient using an implantable medical device (IMD), the method comprising:
detecting activity of a body part of the patient using at least one oscillatory sensor of the IMD, the at least one oscillatory sensor located in an internal thoracic vein (ITV) of the patient;
determining a status of the body part using the IMD based on the activity of the body part; and
monitoring the status of the body part using the IMD by obtaining outputs from the oscillatory sensor.

2. The method of claim 1 further comprising delivering a therapy to the patient with the IMD, wherein the therapy is determined by the IMD using the determined or monitored status of the body part.

3. The method of claim 1 further comprising communicating the status of the body part to an external device, and presenting an indication of the status using the external device.

4. The method of claim 1 wherein the activity is a motion of the heart and the at least one oscillatory sensor is a microphone.

5. The method of claim 1 wherein the activity is motion of the heart and the at least one oscillatory sensor is an accelerometer.

6. The method of claim 1 wherein the activity is heart sound.

7. The method of claim 1 wherein the activity is the fourth heart sound (S₄).

8. The method of claim 1 further comprising detecting activity of the body part using at least a second oscillatory sensor and a third oscillatory sensor which each are also implanted inside the patient, and using outputs of the three oscillatory sensors to spatially triangulate a location of an event identified in the activity of the body part.

9. The method of claim 1 wherein the body part is the heart and the activity comprises heart sounds, and the method further comprises detecting at least one of a cannon wave and a cannon sound, and determining whether atrial fibrillation is occurring based on the presence of cannon waves or cannon sounds.

10. The method of claim 1 wherein the body part is a lung, the activity is breathing, and the at least one oscillatory sensor is a microphone.

11. The method of claim 1 wherein the body part is a lung, the activity is breathing and the at least one oscillatory sensor is an accelerometer.

12. The method of claim 1 wherein the body part is a lung, the activity is breathing, and the IMD is configured to determine the status of the lung by observing at least one of a respiratory interval or a respiratory amplitude.

13. The method of claim 1 wherein the body part is a lung, the activity is breathing, and the IMD is configured to identify a frequency or pattern of vibration indicating wheezing.

14. The method of claim 1 wherein the body part is a lung, the activity is breathing, and the IMD is configured to identify a frequency or pattern of vibration indicating rales.

15. The method of claim 1 wherein the body part is a lung, the activity is breathing, and the IMD is configured to identify a frequency or pattern of vibration indicating snoring.

16. The method of claim 1 wherein the body part is a lung, the activity is breathing, and the IMD is configured to identify a frequency or pattern of vibration indicating rhonchi.

17. The method of claim 1 wherein the body part is a lung, the activity is breathing, and the IMD is configured to identify a pathological asymmetrical respiratory pattern.

18. The method of claim 1 wherein the body part is a lung, the activity is breathing, and the IMD is configured to identify a frequency or pattern of vibration indicating respiratory distress due to one or more of asthma and Chronic Obstructive Pulmonary Disease.

19. A method of treating a patient comprising detecting activity of a body part of a patient using an oscillatory sensor disposed on a lead which is placed in an internal thoracic vein (ITV) of the patient.

20. An implantable medical device comprising a lead having an oscillatory sensor disposed thereon and an implantable canister for coupling to the lead, the implantable canister housing operational circuitry, wherein the lead and oscillatory sensor are configured to be placed in an internal thoracic vein (ITV) of a patient, and the operational circuitry is configured to detect activity of a body part of a patient using the oscillatory sensor while the oscillatory sensor is in the ITV.
